(19) United States
(12) Patent Application Publication
(10) Pub. No.: US 2020/0261751 A1
(43) Pub. Date: Aug. 20, 2020

(54) 3-DIMENSIONAL NASAL FILTER POD

(71) Applicant: Resprana, Inc., New York, NY (US)

(72) Inventors: Sophie Frank, San Francisco, CA (US); Marc-Aurelien Vivant, Brooklyn, NY (US); Micah Steiger, New York, NY (US); Sukanya Goswami, Brooklyn, NY (US); Jai Rathore, Madison, WI (US); Andrea McDonald, New York, NY (US)

(21) Appl. No.: 16/794,660

(22) Filed: Feb. 19, 2020

Related U.S. Application Data
(60) Provisional application No. 62/807,793, filed on Feb. 20, 2019.

(51) Int. Cl. A62B 23/06; A61L 9/04; B01D 69/10; B01D 71/18; B01D 71/38; B01D 71/50; B01D 71/30; B01D 71/54; B01D 71/42

(52) U.S. Cl. CPC A62B 23/06; A61L 9/048; B01D 69/10; B01D 71/18; B01D 71/38; B01D 71/50; B01D 2323/39; B01D 71/54; B01D 71/42; B01D 2325/48; B01D 2325/26; B01D 2325/02; B01D 71/30

(57) ABSTRACT

A 3-dimensional (3D) nasal pod structure is described having a filter that fits in the nasal cavity to capture particles such as pollen, allergens, pollution particles present in air when breathing through the nose.

### CLAIM OF PRIORITY

This application claims priority under 35 U.S.C. § 119 to U.S. provisional application No. 62/807,793 filed on Feb. 20, 2019 in the United States Patent and Trademark Office, the entire disclosure of which is incorporated herein by reference.

### FIELD OF THE EMBODIMENTS

This invention relates to a 3-dimensional nasal filter pod for capturing particles present in air inhaled through the nose.

### BACKGROUND OF THE EMBODIMENTS

The prevalence of chronic inflammation disorders of the lungs is on the increase. For example, in the U.S. alone, about 1 in 12 people or about 25 million suffer from asthma, of which 6.3 million are children. Indeed, asthma is the third-ranking cause of hospitalizations for children under the age of 15. It is now well established that exposure to airborne particles such as animal dander, dust mites, cockroach particles, pollen, tobacco smoke, air pollution, and chemical irritants can trigger an asthma episode or attack resulting in coughing, wheezing, chest tightness, and difficult breathing. Both long-term and short-term exposure can cause health problems such as reduced lung function and more asthma attacks. In the most severe cases, asthma causes the death of more than 3,600 Americans each year.

There is an on-going urgent need for devices and methods that reduce the incidence of or exasperation of chronic respiratory inflammatory disorders.

Examples of related art are summarized below.

U.S. Pat. No. 2,057,397 discloses a nasal inhaler consisting of a cone or cup with an entry and exit and a filler of absorbent material such as cotton or wool inserted into the shell.

U.S. Pat. No. 5,568,808 discloses a disposable nose filter to be inserted in a nostril, including a flexible housing, a filter component and a flutter valve. The nose filter is adapted to be easily inserted and removed from the nostril. The flutter valve forms a seal with the lower exterior portion of the nostril, thus, forcing air through the filter and preventing air from passing between the housing and the inner walls of the nostril.

U.S. Pat. No. 7,354,467 discloses a filtering assembly for nasal cavities including a connector with two rings connected by a link, and two filtering members securely connected to the two rings. The two filtering members are inserted into the wearer's nasal cavities so as to effectively filter foreign objects from entering to the wearer's lungs.

U.S. Pat. No. 7,918,225 discloses a nasal air filtration device comprising a pair of concave-convex filters, and a support structure incorporating a pair of generally annular bases for supporting the filters, and a bridge that couples the bases, to maintain them in a desired spaced-apart relation and to determine a desired angular relationship between the bases and between the associated filters.

U.S. Pat. No. 8,479,735 discloses a nasal cavity filter having a substantially cylindrical supporting wall which adheres to the walls of a nasal cavity, and an inner surface of which defines a cavity for the passage of air inhaled and exhaled by the user. The cavity houses a number of fins, which generate turbulence in the air flowing through, and the surface of which impacted by the air retains particles present in the air.

The published U.S. Patent Application 2018/0104518 discloses a nasal cavity insertion type mask for filtering various kinds of fine dust contained in external air inhaled through the nose.

None of the art described above addresses all of the issues that the present invention does.

For example, none of the references disclose a device comprising a porous membrane disposed over a support structure defining a three-dimensional hollow shape configured to be inserted into a user's nostril.

### SUMMARY OF THE EMBODIMENTS

In a first aspect, a device for preventing the inhalation of airborne particles through a user's nose is disclosed comprising a porous continuous membrane disposed over a support structure defining a three-dimensional hollow shape configured to be inserted into a user's nostril.

In a second aspect, a device for preventing the inhalation of airborne particles through a user's nose is disclosed consisting of a porous continuous membrane disposed over a support structure defining a three-dimensional hollow shape configured to be inserted into a user's nostril.

In certain embodiments of the first or second aspect, the membrane is continuous.

In certain embodiments of the first or second aspect, the device's membrane comprises pores having a diameter of about 50-500 microns.

In certain embodiments of the first or second aspect, the device's membrane comprises pores having a diameter of about 1-25 microns.

In certain embodiments of the first or second aspect, the device's membrane comprises pores having a diameter of about 1-10 microns. In other embodiments the device's membrane comprises pores having a diameter of about a micron.

In certain embodiments of the first or second aspect, the device's membrane comprises pores having a diameter of about 50-300 nanometers.

In certain embodiments of the first or second aspect, the device's membrane comprises pores having a diameter of about a 0.1-5 millimeters.

In certain embodiments of the first or second aspect, the inhaled air comprises allergens, dust particles, viruses, pollution particles and/or airborne particles resulting from a fire.

In certain embodiments of the first or second aspect, the support structure defines a predominantly cylindrical shape with geometric details added to the cylinder perimeter.

In certain embodiments of the first or second aspect, the membrane can be made of natural and/or synthetic fibers. In certain embodiments, the synthetic fibers may comprise, for example, at least one of polyethylene terephthalate (PET), polyethersulfone (PES), polyvinylidene (PVDF), polytetrafluoroethylene (PTFE, Teflon), mixed-cellulose esters (MCE), polyimide (nylon), natural fibers, carbon fibers, or activated carbon, cellulose acetate, cellulose nitrate (collodion), mixed-cellulose esters (MCE, i.e., a mixture of cellulose nitrate/acetate fibers), polycarbonate, polypropylene (PP), polyacrylonitrile (PAN), polymethyl methacrylate (PMMA), polyvinyl alcohol (PVA), polyurethane (PU) and polyvinylchloride (PVC).

In certain embodiments of the first or second aspect, the membrane has electrostatic properties.

In certain embodiments of the first or second aspect, the membrane has antimicrobial properties.

In certain embodiments of the first or second aspect, the membrane comprises silver ions and/or silver nanoparticles to increase an antimicrobial effect against bacteria and viruses.

In certain embodiments of the first or second aspect, the membrane is fabricated by electrospin blowing, fiber weaving, foam casting, injection molding, compression molding or vacuum forming.

In certain embodiments of the first or second aspect, the membrane is produced or replaced by utilizing hydrogel and aqueous gels that have filtering capacity based on ion-exchange properties of the gels

In a third aspect a method for preventing the inhalation of airborne particles through a user's nose comprising inserting the device of the first or second aspect into each of a user's nostril.

### BRIEF DESCRIPTION OF THE DRAWINGS

FIGS. 1A and 1B show exemplary perspective views of certain embodiments of a 3-dimensional nasal filter pod.

### DESCRIPTION OF THE PREFERRED EMBODIMENTS

The preferred embodiments of the present invention will now be described with reference to the drawings. Identical elements in the various figures are identified with the same reference numerals.

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

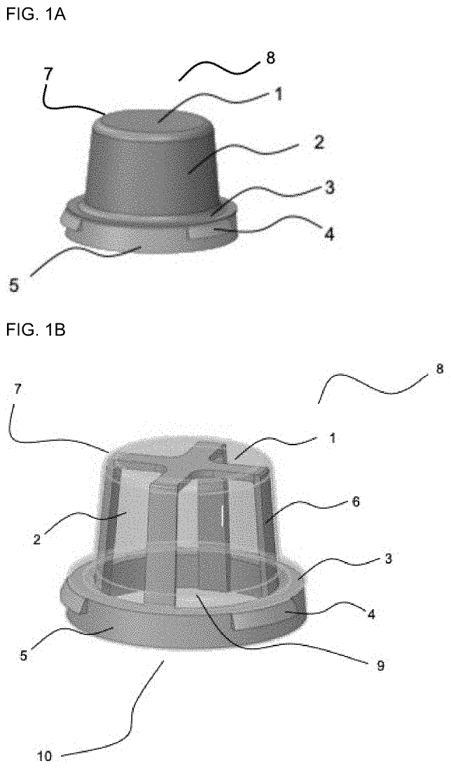

FIG. 1A depicts an exemplary perspective view of a 3-dimensional nasal filter pod 8 having a top 1, side 2, bottom entrance 10 and an interior cavity 9. The porous membrane 7 can have a flange 3 that attaches to a circular support base 5. Protrusions 4 at the periphery of the circular support base 5 help to keep the nasal filter pod in place after insertion into the nasal cavity.

FIG. 1B shows an exemplary perspective view of a 3-dimensional nasal filter pod 8 showing the support structure 6 beneath the porous membrane 7.

In certain embodiments, attachment of the porous membrane 7 to the support base 5 may include, but is not limited to, press fit, slip fit, adhesive, threading or magnetic attachment.

In certain embodiments, the porous membrane 7 can be manufactured with an electro spin blowing manufacturing process to form a flat 2D membrane that can be molded into a 3-dimensional structure.

In certain embodiments, the porous membrane 7 can be manufactured by fiber weaving, foam casting, injection molding, compression molding, clamps, double-sided tape, heat-seal, high-frequency welding or vacuum forming.

In certain embodiments, the porous membrane 7 comprises natural and/or synthetic fibers. In certain embodiments, the synthetic fibers may comprise, for example, at least one of polyethylene terephthalate (PET), polyethersulfone (PES), polyvinylidene (PVDF), polytetrafluoroethylene (PTFE, Teflon), mixed-cellulose esters (MCE), polyamide (nylon), natural fibers, carbon fibers, or activated carbon, cellulose acetate, cellulose nitrate (collodion), mixed-cellulose esters (MCE, i.e., a mixture of cellulose nitrate/acetate fibers), polycarbonate, polypropylene (PP), polyacrylonitrile (PAN), polymethyl methacrylate (PMMA), polyvinyl alcohol (PVA), polyurethane (PU) and polyvinylchloride (PVC). In certain embodiments, the porous membrane 7 may comprise chitosan, collagen, polylactic acid (PLA) and/or Poly(lactic-co-glycolic acid) (PLGA).

In certain embodiments, the porous membrane 7 can include activated carbon conferring electrostatic charge to the membrane.

In certain embodiments, the porous membrane 7 can include silver ions and silver nanoparticles to increase the antimicrobial effect of the overall filtration against bacteria and viruses.

In certain embodiments, the inhaled air comprises allergens, dust particles, viruses, pollution particles such as the ones classified under the particulate matter, pm2.5 denomination, and/or airborne particles resulting of combustion such as the ones resulting from forest fires.

In certain embodiments of the first or second aspect, the membrane is produced or replaced by utilizing hydrogel and aqueous gels that have filtering capacity based on ion-exchange properties of the gels.

In certain embodiments, the porous membrane 7 can have a 3-dimensional (3D) structure, in that it is not limited to a single shape that is cut-out, formed or created as part of or from a two (2) dimensional substrate, such as sheet of paper or a sheet of foam.

In certain embodiments, the porous membrane 7 can have a 3-dimensional shape that creates a maximum surface area for air to flow through, thus decreasing the pressure drop between the air pressure at the entrance of the nasal filter 10 and the pressure at the top 1 of the nasal filter pod 8.

In certain embodiments, the 3-dimensional shape defines a hollow cavity.

In certain embodiments, the nasal filter pod 8 does not contain a filler material such as a sponge or cotton wool.

In certain embodiments, the structural integrity of the nasal filter pod 8 can be re-enforced by a support structure 6 on which the porous membrane 7 can be seated or inserted, stuffed, trapped, encapsulated, merged, woven, or other any means of attachment.

In certain embodiments, methods of attaching the porous membrane 7 to the support structure 6 and/or support 5 include, but are not limited to, gluing, bonding, heat staking, melting, friction welding, vibration welding, sonic welding or insert molding (also referred to as over molding).

In certain embodiment, the porous membrane 7 has pores with an average diameter of about 1, 2, 3, 4, 5, 6, 7, 8, 9, 10, 15, 20 or 25 microns.

In certain embodiment, the porous membrane 7 has pores with an average diameter ranging from 50 to 300 nm.

In certain embodiment, the porous membrane 7 has pores with an average diameter ranging 0.1 to 5 mm.

In certain embodiment, the porous membrane 7 can have a thickness of about 0.1, 0.2, 0.3, 0.4, 0.5, 0.6, 0.7, 0.8, 0.9, 1, 2, 3, 4, 5, 6, 7, 8, 9, or 10 mm.

In certain embodiments, the porous membrane 7 is continuous, e.g., made from a single piece of material. Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A device for preventing the inhalation of airborne particles through a user's nose, said device comprising a porous continuous membrane disposed over a support structure defining a three-dimensional hollow shape configured to be inserted into a user's nostril.

2. The device of claim 1, further comprising: a porous continuous membrane disposed over a support structure defining a three-dimensional hollow shape configured to be inserted into a user's nostril.

3. The device of claim 1, wherein the membrane is continuous.

4. The device of claim 1, wherein the membrane comprises pores having a diameter of about 1-25 microns.

5. The device of claim 1, wherein the membrane comprises pores having a diameter of about 50-500 microns.

6. The device of claim 1, wherein the membrane comprises pores having a diameter of about 50-300 nanometers.

7. The device of claim 1, wherein the membrane comprises pores having a diameter of about 0.1-5 mm.

6. The device of claim 1, wherein the inhaled air comprises particles allergens, dust particles, viruses, pollution particles and/or airborne particles resulting from a fire.

7. The device of claim 1, wherein the support structure defines a cylindrical shape.

8. The device of claim 1, wherein the membrane comprises natural fibers and/or synthetic fibers, said synthetic fibers comprising at least one of polyethylene terephthalate (PET), polyethersulfone (PES), polyvinylidene (PVDF), polytetrafluoroethylene (PTFE, Teflon), polyamide (nylon), carbon fibers, activated carbon, cellulose acetate, cellulose nitrate (collodion), mixed-cellulose esters (MCE), i.e., a mixture of cellulose nitrate/acetate fibers, polycarbonate, polypropylene (PP), polyacrylonitrile (PAN), polymethyl methacrylate (PMMA), polyvinyl alcohol (PVA), polyurethane (PU) and polyvinylchloride (PVC)

9. The device of claim 1, wherein the membrane has electrostatic properties.

10. The device of claim 1, wherein the membrane has antimicrobial properties.

11. The device of claim 1, wherein the membrane is fabricated by electrospin blowing, fiber weaving, foam casting, injection molding, compression molding or vacuum forming.

12. The device of claim 1, wherein the membrane is produced or replaced by utilizing hydrogel and aqueous gels that have filtering capacity based on ion-exchange properties of the gels.

13. A method for preventing the inhalation of airborne particles through a user's nose comprising; inserting the device of claim 1 into each of a user's nostril.
